(19) United States
(12) Patent Application Publication
(10) Pub. No.: US 2018/0169295 A1
(43) Pub. Date: Jun. 21, 2018

(54) FILM, MANUFACTURING METHOD THEREOF, AND USE THEREOF

(71) Applicant: Industrial Technology Research Institute, Hsinchu (TW)

(72) Inventors: Ming-Chia YANG, Taipei City (TW); Wei-Hong CHANG, Linnei Township (TW); Yun-Han LIN, Taichung City (TW); Kun-Mao KUO, Tainan City (TW); Li-Jie LIN, New Taipei City (TW); Tai-Horng YOUNG, Taipei City (TW)

(73) Assignee: Industrial Technology Research Institute, Hsinchu (TW)

(21) Appl. No.: 15/388,980

(22) Filed: Dec. 22, 2016

(30) Foreign Application Priority Data
Dec. 21, 2016 (TW) 105142419

(51) Int. Cl. A61L 24/04

(52) U.S. Cl. CPC A61L 24/043; A61L 24/046

(57) ABSTRACT

The present disclosure provides a film composed of a polymer mixture, wherein the polymer mixture includes: a hydrophobic composition including polycaprolactone (PCL); and at least one hydrophilic polymer selected from a group consisting of: alginate, gelatin, chitosan, hyaluronic acid, collagen, demineralized bone matrix (DSM), carboxymethyl cellulose (CMC), fibrin, polyoxyethylene, polyethylene glycol (PEG) and polyvinylpyrrolidone, wherein the weight ratio of the hydrophobic composition to the at least one hydrophilic polymer is about 1:0.01-100, and wherein the film has the effect of preventing leakage from a surgical wound or a diffuse wound.

### CROSS REFERENCE TO RELATED APPLICATION

The application is based on, and claims priority from, Taiwan Application Serial Number 105142419, filed on Dec. 21, 2016, the disclosure of which is hereby incorporated by reference herein in its entirety.

### TECHNICAL FIELD

The technical field relates to a film, a manufacturing method thereof, and use thereof.

### BACKGROUND

Tissue adhesives and sealants have many potential medical applications, such as wound closures; as a supplement or substitute for sutures or staples used in surgical operations; to prevent the leakage of fluids such as blood, bile, gastrointestinal fluids and cerebrospinal fluid; or for affixing a surgical mesh to soft tissue. Among adhesives, fibrin is the most widely used. However, fibrin has defects that include slow solidification and poor mechanical strength. Moreover, when using fibrin, there is a risk of viral infection, and tissue adhesion can easily occur on the wound. Thus the use of fibrin is limited in surgical applications.

In cardiovascular surgery or a liver, gallbladder, intestine, or stomach resection, surgical mesh or sutures are usually used to reinforce the part subjected to the surgery, however, the material in common use at present has problems that easily result in foreign body inflammation and the inability to absorb tissue fluid. Furthermore, in a resection for the gastrointestinal tract, the part undergoing the resection may have experience leakage of digestive fluid, body fluid, or blood, which can result in peritonitis, and during the repair process, peristalsis of the organ may occur. Therefore, an attach film has to be capable of perfectly fitting to the positions which are sutured.

However, because surgical mesh has hard material and bad tissue adaptation, it cannot be tightly attached to soft tissue, and thus needs to be affixed using sutures, which can result in inconvenience during surgical operations. In addition, at present, the commercial surgical mesh appliances commonly used for auto-fixing, such as nails or hidden buttons, still often need be enhanced by use of sutures, and thus a secondary infection and leakage can easily occur at the location of the suture.

Therefore, at present, a novel patching film which can achieve a perfect fit and seal in the damp conditions within the body without the need for an additional fixative is needed.

### SUMMARY

The present disclosure provides a film composed of a polymer mixture, wherein the polymer mixture comprises: a hydrophobic composition including polycaprolactone (PCL); and at least one hydrophilic polymer selected from a group consisting of: alginate, gelatin, chitosan, hyaluronic acid, collagen, demineralized bone matrix (DBM), carboxymethyl cellulose (CMC), fibrin, polyoxyethylene, polyethylene glycol (PEG) and polyvinylpyrrolidone, wherein the weight ratio of the hydrophobic composition to the at least one hydrophilic polymer is about 1:0.01-100, and wherein the film has the effect of preventing leakage from a surgical wound or a diffuse wound.

The present disclosure also provides a method for manufacturing a film, comprising: preparing a polymer mixture, wherein a method for preparing the polymer mixture comprises: preparing a hydrophobic solution, wherein a solute of the hydrophobic solution comprises polycaprolactone; and adding at least one hydrophilic polymer as a dispersing agent to the hydrophobic solution and mixing it with the hydrophobic solution, wherein the at least one hydrophilic polymer is selected from a group consisting of alginate, gelatin, chitosan, hyaluronic acid, collagen, demineralized bone matrix (DBM), carboxymethyl cellulose (CMC), fibrin, polyoxyethylene, polyethylene glycol (PEG) and polyvinylpyrrolidone, wherein the weight ratio of the solute of the hydrophobic solution to the at least one hydrophilic polymer is about 1:0.01-100: and drying the polymer mixture to form a film.

The present disclosure also provides a use of the film mentioned above for the manufacture of a laminating film for a surgical wound or a diffuse wound.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows a situational schematic diagram of using a film of the present disclosure in one embodiment;

FIGS. 2A to 2D show thermogravimetric analysis results of the films prepared in Examples 1-1 to 1-4 at different locations;

FIGS. 3A to 3C show thermogravimetric analysis results of the films prepared in Comparative Example 1 and prepared in Example 1 and Example 2 at different locations;

FIG. 4 shows analysis result of Fourier transform infrared spectrometry (FT-IR) of the film prepared in Example 3;

FIG. 5 shows the results of standard test of burst strength for the films prepared in Comparative Example 1 and prepared in Example 2 and Example 3, and the commercial sealing film (TachoSil) and sealing patch (TissePatch);

FIG. 6 shows the results of a standard test of tensile properties for the films prepared in Comparative Example land prepared in Example 2 and Example 3, and the commercial sealing film (TachoSil) and sealing patch (TissePatch);

FIGS. 7A and 7B show photographs of surface structures of the films prepared in Example 3 and prepared in Comparative Example 1;

FIG. 8 shows the full roughness of the surfaces of the films prepared in Comparative Example 1 and prepared in Example 2 and Example 3;

FIG. 9 shows photographs of a surgical wound on the liver of a rat before implanting a film and 14 days after implanting the film, and shows the result of hematoxylin and eosin (H&E) stain for the tissue attached by the film;

FIG. 10 shows photographs of a surgical wound on the stomach of a rat before implanting a film and 14 days after implanting the film, and shows the result of hematoxylin and eosin (H&E) stain for the tissue attached by the film

### DETAILED DESCRIPTION

In one aspect of the present disclosure, a film which is a biodegradable non-fiber form film, and can be well attached on a surgical wound or a diffuse wound without needing sutures or another fixing manner, and can prevent leakage of tissue fluid, is provided. Furthermore, in one aspect of the present disclosure, the present disclosure provides an adherent film which is fixative-free, and this film has the effect of preventing leakage.

In one embodiment, the film of the present disclosure mentioned above may be composed of a polymer mixture, but it is not limited thereto.

The foregoing polymer mixture may comprise, but is not limited to, a hydrophobic composition and at least one hydrophilic polymer.

The above-mentioned hydrophobic composition may comprise polycaprolactone (PCL), but is not limited thereto. The molecular weight of the polycaprolactone may be about 5000-150000. In one embodiment, the molecular weight of the polycaprolactone is about 120000.

In the present disclosure, examples of a suitable hydrophilic polymer may include, but are not limited to, alginate, gelatin, chitosan, hyaluronic acid, collagen, demineralized bone matrix (DBM), carboxymethyl cellulose (CMC), fibrin, polyoxyethylene, polyethylene glycol (PEG), polyvinylpyrrolidone, and combinations thereof.

In one embodiment, the at least one hydrophilic polymer mentioned above may be in the form of a solid particle. In this embodiment, the particle size of the solid particle is about 1-1000 μm, but is not limited thereto.

In another embodiment, the at least one hydrophilic polymer mentioned above may be dissolved in a solvent to be in the form of a liquid polymer. In this embodiment, examples of said solvent may include, but are not limited to, water, ethanol, acetone, an acidic solution, an alkaline solution, and a buffer solution. Moreover, in this embodiment, the limiting viscosity of the liquid polymer may be about 1-200 dl/g, but it is not limited thereto.

In addition, in the film of the present disclosure, the weight ratio of the hydrophobic composition to the at least one hydrophilic polymer may be about 1:0.01-100, but it is not limited thereto. In one embodiment, the weight ratio of the hydrophobic composition to the at least one hydrophilic polymer may be about 1:0.625. In another embodiment, the weight ratio of the hydrophobic composition to the at least one hydrophilic polymer may be about 1:1.25.

In one embodiment, in the film of the present disclosure, the at least one hydrophilic polymer mentioned above is alginate. Moreover, in this embodiment, the weight ratio of the preceding hydrophobic composition to the alginate is about 1:0.05-80, such as 1:0.0625-60, but is not limited thereto. In one specific embodiment, the weight ratio of the preceding hydrophobic composition to the alginate is about 1:0.0625, etc.

In another embodiment, in the film of the present disclosure, the at least one hydrophilic polymer mentioned above is gelatin. In this embodiment, the weight ratio of the foregoing hydrophobic composition to the gelatin is about 1:0.05-80, such as 1:0.0625-60, but it is not limited thereto. In one specific embodiment, the weight ratio of the foregoing hydrophobic composition to the gelatin is about 1:1.25, etc.

In one embodiment of the present disclosure, in the preceding polymer mixture which composes the film of the present disclosure, the hydrophobic composition is composed of polycaprolactone. In this embodiment, the weight ratio of the polycaprolactone to the at least one hydrophilic polymer mentioned above may be about 1:0.05-80, such as 1:0.0625-60, but it is not limited thereto. In one specific embodiment, the weight ratio of the polycaprolactone to the at least one hydrophilic polymer mentioned above may be about 1:1.25, etc.

In another embodiment of the present disclosure, in the preceding polymer mixture which composes the film of the present disclosure, the hydrophobic composition, in addition to polycaprolacton, may further comprise at least one hydrophobic polymer. The hydrophobic polymer mentioned herein may comprise polylactic acid (PLA), poly(lactic-co-glycolic acid) (PLGA), poly(glycolic acid) (PGA), polyhydroxybutyrate, (PHB), polydioxanone (PDS), poly(propylene fumarate) (PPF), polyanhydrides, polyacetals, poly(ortho esters), polycarbonates, polyurethanes, polyphosphazenes, polyphosphoester), a combination thereof, etc., but it is not limited thereto. In the hydrophobic composition mentioned above, the weight ratio of the polycaprolactone to the at least one hydrophilic polymer mentioned above may be about 1:0.01-10, such as 1:0.25, but it is not limited thereto. Furthermore, in this embodiment, the weight ratio of the hydrophobic composition mentioned above to the at least one hydrophilic polymer mentioned above may be about 1:0.05-80, such as 1:0.0625-60, but it is not limited thereto. In one specific embodiment, the weight ratio of the hydrophobic composition mentioned above to the at least one hydrophilic polymer mentioned above may be about 1:0.0625, 1:0.625, 1:1.25, 1:20 or 1:60. Moreover, in this embodiment, the weight ratio of the polycaprolactone to the hydrophilic polymer mentioned above may be about 1:0.02-90, such as 1:0.07-75, but it is not limited thereto. In one specific embodiment, the weight ratio of the polycaprolactone to the at least one hydrophilic polymer mentioned above may be about 1:0.078125, 1:0.78125, 1:1.1.5625, 1:31.25 or 1:75, etc.

In the embodiment in which the hydrophobic composition, in addition to polycaprolacton, may further comprise at least one hydrophobic polymer, the molecular weight of the polycaprolactone may be about 5000-150000, but it is not limited thereto. Furthermore, in this embodiment, the weight ratio of the hydrophobic composition to the at least one hydrophilic polymer may be about 1:0.05-80, but it is not limited thereto.

In addition, in the embodiment in which the hydrophobic composition, in addition to polycaprolacton, may further comprise at least one hydrophobic polymer, the at least one hydrophilic polymer mentioned above may be in the form of a solid particle, and the particle size of the solid particle may be about 1-1000 pin, but it is not limited thereto. Alternatively, in this embodiment, the at least one hydrophilic polymer may be dissolved in a solvent to be in the form of liquid polymer, and examples of the solvent may include, but are not limited to, water, ethanol, acetone, an acidic solution, an alkaline solution, and a buffer solution. In addition, in this embodiment, the limiting viscosity of said liquid polymer may be about 1-200 dl/g, but it is not limited thereto.

Furthermore, in the embodiment in which the hydrophobic composition, in addition to polycaprolacton, may further comprise at least one hydrophobic polymer, in the preceding polymer mixture, the at least one hydrophilic polymer mentioned above is alginate, and the weight ratio of the hydrophobic composition to the alginate is about 1:0.05-80, but is not limited thereto. Alternatively, in this embodiment, the at least one hydrophilic polymer is gelatin, and the weight ratio of the hydrophobic composition to the gelatin is about 1:0.05-80, but it is not limited thereto.

Moreover, in one embodiment, in the preceding polymer mixture which composes the film of the present disclosure, the hydrophobic composition, in addition to polycaprolacton, may further comprise at least one hydrophobic polymer, and the at least one hydrophobic polymer may be poly(lactic-co-glycolic acid). In this embodiment, the weight ratio of the hydrophobic composition to the at least one hydrophilic polymer may be about 1:0.05-80, such as 1:0.0625-60, but is not limited thereto. In one specific embodiment, the weight ratio of the hydrophobic composition to the at least one hydrophilic polymer mentioned above may be about 1:0.0625, 1:0.625, 1:1.25, 1:20 or 1:60. Furthermore, in this embodiment, the weight ratio of the polycaprolactone to the at least one hydrophilic polymer mentioned above may be about 1:0.02-90, such as 1:0.07-75, but it is not limited thereto. In one specific embodiment, the weight ratio of the polycaprolactone to the at least one hydrophilic polymer mentioned above may be about 1:0.078125, 1:0.78125, 1:1.1.5625, 1:31.25 or 1:75, etc.

In the embodiment in which the hydrophobic composition, in addition to polycaprolacton, may further comprise poly(lactic-co-glycolic acid), the molecular weight of the polycaprolactone may be about 5000-150000, such as 120000, but it is not limited thereto.

Furthermore, in the embodiment in which the hydrophobic composition, in addition to polycaprolacton, may further comprise poly(lactic-co-glycolic acid), the at least one hydrophilic polymer may be in the form of a solid particle, and the particle size of the solid particle is about 1-1000 μm, but is not limited thereto. Alternatively, in this embodiment, the at least one hydrophilic polymer may be dissolved in a solvent to be in the form of liquid polymer, and examples of said solvent may include, but are not limited to, water, ethanol, acetone, an acidic solution, an alkaline solution, and a buffer solution, and the limiting viscosity of the liquid polymer may be about 1-200 dl/g, but it is not limited thereto.

In the preceding embodiment in which the hydrophobic composition, in addition to polycaprolacton, may further comprise poly(lactic-co-glycolic acid), in one specific embodiment, in the film of the present disclosure, the at least one hydrophilic polymer mentioned above is alginate, and the weight ratio of the hydrophobic composition mentioned above to the alginate is about 1:0.05-80, such as, about 1:0.0625, 1:0.625, 1:1.25, 1:20 or 1:60, but is not limited thereto. In addition, in another specific embodiment, in the film of the present disclosure, the at least one hydrophilic polymer mentioned above is gelatin. In this embodiment, the weight ratio of the hydrophobic composition mentioned above to the gelatin is about 1:0.05-80, such as 1:1.25, but it is not limited thereto.

In another aspect of the present disclosure, a method for manufacturing a film is provided, wherein said film is a non-fiber form film, and can be attached on a surgical wound or a diffuse wound without the need for sutures or any other fixing manner, and can prevent leakage of tissue fluid.

In one embodiment, the method for manufacturing a film mentioned above may comprise the following steps, but it is not limited thereto.

First, a polymer mixture is prepared.

Next, the polymer mixture is dried to form a film.

Moreover, a method for preparing the preceding polymer mixture may comprise the following steps, but is not limited thereto.

First, a hydrophobic solution is prepared, and a solute of the hydrophobic solution may comprise, but is not limited to, polycaprolactone. The molecular weight of the polycaprolactone may be about 5000-150000, but is not limited thereto. In one embodiment, the molecular weight of the polycaprolactone may be 120000. Furthermore, in one embodiment, the hydrophobic solution is formed by dissolving the polycaprolactone in a solvent. Examples of the solvent mentioned above may include acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene, and mixture solutions thereof, but it is not limited thereto.

Next, at least one hydrophilic polymer as a dispersing agent is added to the hydrophobic solution and mixed with the hydrophobic solution. The weight ratio of the solute of the foregoing hydrophobic solution to the at least one hydrophilic polymer mentioned above may be about 1:0.01-100, but it is not limited thereto. In one embodiment, the weight ratio of the solute of the hydrophobic solution to the at least one hydrophilic polymer may be about 1:0.625. In another embodiment, the weight ratio of the solute of the hydrophobic solution to the at least one hydrophilic polymer may be about 1:1.25.

Examples of suitable hydrophilic polymer may include, but are not limited to, alginate, gelatin, chitosan, hyaluronic acid, collagen, demineralized bone matrix (DBM), carboxymethyl cellulose (CMC), fibrin, polyoxyethylene, polyethylene glycol (PEG), polyvinylpyrrolidone, and combinations thereof.

In one embodiment, the at least one hydrophilic polymer may be in the form of a solid particle. In this embodiment, the particle size of the solid particle is about 1-1000 μm, but it is not limited thereto.

In another embodiment, the at least one hydrophilic polymer may be dissolved in a solvent to be in the form of liquid polymer. In this embodiment, examples of said solvent may include, but are not limited to, water, ethanol, acetone, an acidic solution, an alkaline solution, and a buffer solution. Furthermore, in this embodiment, the limiting viscosity of the liquid polymer may be about 1-200 dl/g, but it is not limited thereto.

In one embodiment, the at least one hydrophilic polymer mentioned above is alginate. Moreover, in this embodiment, the weight ratio of the solute of the foregoing hydrophobic solution to the alginate may be about 1:0.05-80, such as 1:0.0625-60, but is not limited thereto. In one specific embodiment, the weight ratio of the solute of the foregoing hydrophobic solution to the alginate may be about 1:0.0625, etc.

In another embodiment, the at least one hydrophilic polymer mentioned above is gelatin. In this embodiment, the weight ratio of the solute of the preceding hydrophobic solution to the gelatin may be about 1:0.05-80, such as 1:0.0625-60, but is not limited thereto. In one specific embodiment, the weight ratio of the solute of the preceding hydrophobic solution to the gelatin may be about 1:1.25, etc.

In one embodiment, in the polymer mixture, the solute of the hydrophobic solution, in addition to polycaprolacton, may further comprise at least one hydrophobic polymer. The hydrophobic polymer mentioned herein may comprise polylactic acid (PLA), poly(lactic-co-glycolic acid) (PLGA), poly(glycolic acid) (PGA), polyhydroxybutyrate (PHB), polydioxanone (PDS), poly(propylene fumarate) (PPF), polyanhydrides, polyacetals, poly(ortho esters, polycarbonates, polyurethanes, polyphosphazenes, polyphosphoester, or a combination thereof, but it is not limited thereto. In the solute of the hydrophobic solution, the weight ratio of the polycaprolactone to the at least one hydrophilic polymer mentioned above may be about 1:0.01-10, such as 1:0.25, but it is not limited thereto. Furthermore, in this embodiment, the weight ratio of the solute of the preceding hydrophobic solution to the at least one hydrophilic polymer mentioned above may be about 1:0.05-80, such as 1:0.0625-60, but it is not limited thereto. In one specific embodiment, the weight ratio of the solute of the preceding hydrophobic solution to the at least one hydrophilic polymer mentioned above may be about 1:0.0625, 1:0.625, 1:1.25, 1:20 or 1:60. In addition, in this embodiment, the weight ratio of the polycaprolactone to the at least one hydrophilic polymer mentioned above may be about 1:0.02-90, such as 1:0.07-75, but it is not limited thereto. In one specific embodiment, the weight ratio of the polycaprolactone to the at least one hydrophilic polymer mentioned above may be about 1:0.078125, 1:0.78125, 1:1.1.5625, 1:31.25 or 1:75, etc.

In the embodiment in which the solute of the hydrophobic solution, in addition to polycaprolacton, may further comprise at least one hydrophobic polymer, the hydrophobic solution may be formed by a method, and the method may comprise dissolving the polycaprolactone in a first solvent to form a first solution, and dissolving the at least one hydrophobic polymer in a second solvent to form a second solution, and then mixing the first solution with the second solution to form the hydrophobic solution. Furthermore, the first solvent and the second solvent may be the same or different.

Examples of the foregoing first solvent may include acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene, and combinations thereof, but they are not limited thereto. The second solvent mentioned above may comprise, but is not limited to, acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene or a combination thereof.

Alternatively, in the embodiment in which the solute of the hydrophobic solution, in addition to polycaprolacton, may further comprise at least one hydrophobic polymer, the hydrophobic solution may be formed by another method, and the method may comprise dissolving the polycaprolactone and the at least one hydrophobic polymer mentioned above in the same solvent to form said hydrophobic solution. The solvent mentioned herein may comprise acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene or a combination thereof, but it is not limited thereto.

Moreover, in the embodiment in which the solute of the hydrophobic solution, in addition to polycaprolacton, may further comprise at least one hydrophobic polymer, the molecular weight of the polycaprolactone may be about 5000-150000, but is not limited thereto. In addition, in this embodiment, the weight ratio of the solute of the hydrophobic solution to the at least one hydrophilic polymer may be about 1:0.05-80, but is not limited thereto.

Furthermore, in the embodiment in which the solute of the hydrophobic solution, in addition to polycaprolactone, may further comprise at least one hydrophobic polymer, the at least one hydrophilic polymer mentioned above may be in the form of a solid particle, and the particle size of the solid particle may be about 1-1000 μm, but is not limited thereto. Alternatively, in this embodiment, the at least one hydrophilic polymer mentioned above may be dissolved in a solvent to be in the form of liquid polymer, and examples of said solvent may include, but are not limited to, water, ethanol, acetone, an acidic solution, an alkaline solution, and a buffer solution. In addition, in this embodiment, the limiting viscosity of the liquid polymer may be about 1-200 dl/g, but it is not limited thereto.

Moreover, in the preceding embodiment in which the solute of the hydrophobic solution, in addition to polycaprolacton, may further comprise at least one hydrophobic polymer, in the polymer mixture mentioned above, the at least one hydrophilic polymer mentioned above is alginate, and the weight ratio of the solute of the hydrophobic solution to the alginate may be about 1:0.05-80, but is not limited thereto. Alternatively, in this embodiment, the at least one hydrophilic polymer is gelatin, and the weight ratio of the solute of the hydrophobic solution to the gelatin may be about 1:0.05-80, but is not limited thereto.

In addition, in one embodiment, in the polymer mixture, the solute of the hydrophobic solution, in addition to polycaprolacton, may further comprise at least one hydrophobic polymer, and the at least one hydrophobic polymer may be poly(lactic-co-glycolic acid). In this embodiment, the weight ratio of the solute of the hydrophobic solution to the at least one hydrophilic polymer may be about 1:0.05-80, such as 1:0.0625-60, but is not limited thereto. In one specific embodiment, the weight ratio of the solute of the hydrophobic solution to the at least one hydrophilic polymer mentioned above may be about 1:0.0625, 1:0.625, 1:1.25, 1:20 or 1:60. Moreover, in this embodiment, the weight ratio of the polycaprolactone to the at least one hydrophilic polymer mentioned above may be about 1:0.02-90, such as, 1:0.07-75, but is not limited thereto. In one specific embodiment, the weight ratio of the polycaprolactone to the at least one hydrophilic polymer mentioned above may be about 1:0.078125, 1:0.78125, 1:1.1.5625, 1:31.25 or 1:75, etc.

In the preceding embodiment in which the solute of the hydrophobic solution, in addition to polycaprolacton, may further comprise poly(lactic-co-glycolic acid), the molecular weight of the polycaprolactone may be about 5000-150000, such as 120000, but it is not limited thereto.

Moreover, in the preceding embodiment in which the solute of the hydrophobic solution, in addition to polycaprolacton, may further comprise poly(lactic-co-glycolic acid), the at least one hydrophilic polymer may be in the form of a solid particle, and the particle size of the solid particle is about 1-1000 μm, but it is not limited thereto. Alternatively, in this embodiment, the at least one hydrophilic polymer may be dissolved in a solvent to be in the form of a liquid polymer, and examples of said solvent may include, but are not limited to, water, ethanol, acetone, an acidic solution, an alkaline solution, and a buffer solution, and the limiting viscosity of the liquid polymer may be about 1-200 dl/g, but it is not limited thereto.

In the preceding embodiment in which the solute of the hydrophobic solution, in addition to polycaprolacton, may further comprise poly(lactic-co-glycolic acid), in one specific embodiment, in the film of the present disclosure, the at least one hydrophilic polymer mentioned above is alginate, and the weight ratio of the solute of the hydrophobic solution mentioned above to the alginate may be about 1:0.05-80, such as about 1:0.0625, 1:0.625, 1:1.25, 1:20 or 1:60, but is not limited thereto. Moreover, in another specific embodiment, in the film of the present disclosure, the at least one hydrophilic polymer mentioned above is gelatin. In this embodiment, the weight ratio of the solute of the preceding hydrophobic solution to the gelatin may be about 1:0.05-80, such as 1:1.25, but is not limited thereto.

It should be noted that in the method for manufacturing a film of the present disclosure, by using the at least one hydrophilic polymer as a dispersing agent, the ingredients of the polymer mixture can be uniformly distributed, and a film with an even surface can be formed.

In addition, in the method for manufacturing a film of the present disclosure, a manner for drying the polymer mixture has no particular limitation, only if the polymer mixture is able to form a film. In one embodiment, the polymer mixture may be poured on a plate, and then scraped with a scraper to perform a film scraping procedure, and after that, dried to form a film.

Furthermore, in one embodiment, the method for manufacturing a film of the present disclosure may further comprise performing a film stripping process after the polymer mixture is dried to form a film.

The film stripping process mentioned above has no particular limitation, only if the film can be detached from the material it attaches to. In one embodiment, the film stripping process comprises immersing the film with the material it attaches to in a film-stripping solution to make the film detach from the material it attaches to.

Examples of the film-stripping solution may include, but are not limited to, ethanol, glycerol, soap base, and polyethylene glycol.

Moreover, the weight ratio of the film to the film-stripping solution may be about 1-20:10-2000, but it is not limited thereto.

In another aspect of the present application, a film which is manufactured by any one of the preceding methods for manufacturing a film of the present disclosure is provided.

In one embodiment, any film of the present application mentioned above may have a thickness of about 1-3000 μm. Moreover, in one embodiment, the full roughness (Rz) of any film of the present application mentioned above may be about 1-100 μm.

Furthermore, the burst pressure of any film of the present application mentioned above may be about 5-1000 cm-H₂O.

In addition, the tensile strength of any film of the present application mentioned above may be about 5-3000 kPa.

Any film of the present application mentioned above is biodegradable, and can be used for reinforcing sutures and preventing leakage in a surgical wound, and can laminate to a tissue by itself without the need of a suture.

Besides, any film of the present application mentioned above can have an isolating effect by attachment while being implanted to a soft tissue or an organ in a common surgery. In addition, any film of the present application mentioned above is capable of preventing exudation of tissue fluid and reinforcing a frail part of a soft tissue. For example, it can be used for patching in a cardiovascular surgery, for patching in a liver, gall, gastrointestinal endoscopy or patching other organs or anadesma without being fixed with surgical sutures.

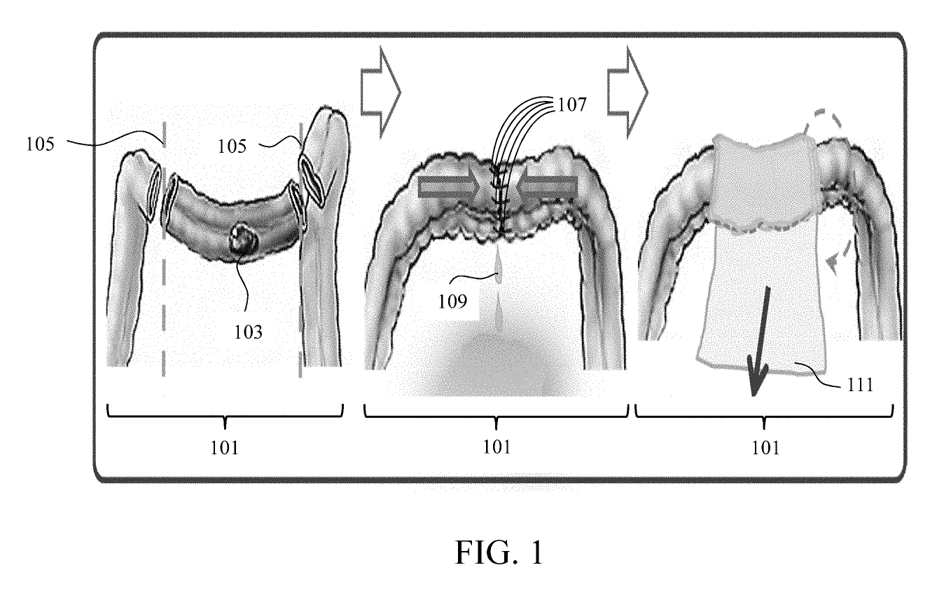

In one embodiment, a situation in which any film of the present application mentioned above may be used is shown as FIG. 1. A section containing a lesion 103 of a large intestine 101 is excised through excising lines 105. After that, two ends at the excising section of the remaining large intestine are sutured by sutures 107, however, leakage 109 may still occur at the suturing position. Therefore, the suturing position is attached or twined round by a film of the present disclosure to achieve the effect of preventing leakage.

Therefore, in another aspect of the present disclosure, a use for any film of the present application mentioned above for manufacturing a laminating film for surgical wounds and diffuse wounds is provided. The laminating film for surgical wounds and diffuse wounds can attach to tissue by itself without needing to be fixed with an external force.

### EXAMPLES

### A. Preparation of Films

### 1. Comparative Example

### Comparative Example 1: Preparation of polycaprolactone/poly(lactic-co-glycolic acid) (PCL/PLGA) film

1. 3.2±0.05 g of polycaprolactone (Mw. 120K) was added to 10 ml of dichloromethane (DCM), and then mixed with it at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed with it at 50 rpm for 3 hours to form a poly(lactic-co-glycolic acid) solution.

3. The polycaprolactone solution and the poly(lactic-co-glycolic acid) solution were equal in proportion and mixed to form a mixture and continuously stirred.

4. After stirring for about 1 minute±10 seconds, the mixture was poured on a teflon plate, and scraped with a 300 μm scraper to perform a film scraping procedure, and after that, left to stand in a fume hood overnight to form a film.

5. The film was removed from the teflon plate to complete the preparation of a polycaprolactone/poly(lactic-co-glycolic acid)(PCL/PLGA) film.

### 2. Examples

### Example 1: Preparation of Polycaprolactone/Poly(Lactic-Co-Glycolic Acid)/Alginate (PCL/PLGA/AA) Films

### Example 1-1: Preparation of a Film in which the Ratio of (Polycaprolactone/Poly(Lactic-Co-Glycolic Acid)) to Alginate was 1:0.0625

1. 3.2±0.05 g of polycaprolactone (Mw. 120K) was added to 10 ml of dichloromethane (DCM), and then mixed with it at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed with it at 50 rpm for 3 hours to form a poly(lactic-co-glycolic acid) solution.

3. The polycaprolactone solution and the poly(lactic-co-glycolic acid) solution were uniformly mixed to form a mixture solution.

4. 0.25 g of alginate (AA) was added to the mixture solution to form a mixture and continuously stirred.

5. After stirring for about 1 minute±10 seconds, the mixture was poured on a teflon plate, and scraped with a 300 μm scraper to perform a film scraping procedure, and after that, left to stand in a fume hood overnight to form a film.

6. The film was removed from the teflon plate.

7. The film was washed 4 times with 2 L deionized water for 1 hour.

8. After washing, the film was placed in a 37° C. oven for drying for 16-24 hours to complete the preparation of polycaprolactone/poly(lactic-co-glycolic acid)/alginate (PCL/PLGA/AA) film of Example 1-1.

### Example 1-2: Preparation of a Film in which the Ratio of (Polycaprolactone/Poly(Lactic-Co-Glycolic Acid)) to Alginate was 1:0.625

1. 3.2±0.05 g of polycaprolactone (Mw. 120K) was added to 10 ml of dichloromethane (DCM), and then mixed with it at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed with it at 50 rpm for 3 hours to form a poly(lactic-co-glycolic acid) solution.

3. The polycaprolactone solution and the poly(lactic-co-glycolic acid) solution were uniformly mixed to form a mixture solution.

4. 2.5 g of alginate (AA) was added to the mixture solution to form a mixture and continuously stirred.

5. After stirring for about 1 minute±10 seconds, the mixture was poured on a teflon plate, and scraped with a 300 μm scraper to perform a film scraping procedure, and after that, left to stand in a fume hood overnight to form a film.

6. The film was removed from the teflon plate.

7. The film was washed 4 times with 2 L deionized water for 1 hour.

8. After washing, the film was placed in a 37° C. oven for drying for 16-24 hours to complete the preparation of polycaprolactone/poly(lactic-co-glycolic acid)/alginate (PCL/PLGA/AA) film of Example 1-2.

### Example 1-3: Preparation of a Film in which the Ratio of (Polycaprolactone/Poly(Lactic-Co-Glycolic Acid)) to Alginate was 1:20

1. 3.2±0.05 g of polycaprolactone (Mw. 120K) was added to 10 ml of dichloromethane (DCM), and then mixed with it at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed with it at 50 rpm for 3 hours to form a poly(lactic-co-glycolic acid) solution.

3. The polycaprolactone solution and the poly(lactic-co-glycolic acid) solution were uniformly mixed to form a mixture solution.

4. 100 g of alginate (AA) was added to the mixture solution to form a mixture and continuously stirred.

5. After stirring for about 1 minute±10 seconds, the mixture was poured on a teflon plate, and scraped with a 300 μm scraper to perform a film scraping procedure, and after that, left to stand in a fume hood overnight to form a film.

6. The film was removed from the teflon plate.

7. The film was washed 4 times with 2 L deionized water for 1 hour.

8. After washing, the film was placed in a 37° C. oven for drying for 16-24 hours to complete the preparation of polycaprolactone/poly(lactic-co-glycolic acid)/alginate (PCL/PLGA/AA) film of Example 1-3.

### Example 1-4: Preparation of a Film in which the Ratio of (Polycaprolactone/Poly(Lactic-Co-Glycolic Acid)) to Alginate was 1:60

1. 3.2±0.05 g of polycaprolactone (Mw. 120K) was added to 10 ml of dichloromethane (DCM), and then mixed with it at 50 rpm for 3 hours to form a polycaprolactone solution.

2. 0.8±0.05 g of poly(lactic-co-glycolic acid) (PLGA) (Mw. 240K) was added to 10 ml of dichloromethane (DCM), and then mixed with it at 50 rpm for 3 hours to form a poly(lactic-co-glycolic acid) solution.

3. The polycaprolactone solution and the poly(lactic-co-glycolic acid) solution were uniformly mixed to form a mixture solution.

4. 240 g of alginate (AA) was added to the mixture solution to form a mixture and continuously stirred.

5. After stirring for about 1 minute±10 seconds, the mixture was poured on a teflon plate, and scraped with a 300 μm scraper to perform a film scraping procedure, and after that, left to stand in a fume hood overnight to form a film.

6. The film was removed from the teflon plate.

7. The film was washed 4 times with 2 L deionized water for 1 hour.

8. After washing, the film was placed in a 37° C. oven for drying for 16-24 hours to complete the preparation of polycaprolactone/poly(lactic-co-glycolic acid)/alginate (PCL/PLGA/AA) film.

### Example 2: Preparation of Polycaprolactone/Gelatin (PCL/Gelatin) Film

1. 4 g of polycaprolactone (Mw. 120K) was added to 20 ml of dichloromethane (DCM), and then mixed with it at 50 rpm for 3 hours to prepare a 20% polycaprolactone solution.

2. 5 g of gelatin was added to 10 ml of deionized water and heated in a 50° C. oven for 16 hours to be dissolved to prepare a 50% gelatin solution.

3. The 50% gelatin solution was taken out from the oven and poured to the 20% polycaprolactone solution (time for taking the 50% gelatin solution out and pouring it to the 20% polycaprolactone solution had to be in 1 minute) to perform mixing and stirring to form a mixture.

4. After stirring for about 1 minute±10 seconds, the mixture was poured on a teflon plate, and scraped with a 300 μm scraper to perform a film scraping procedure, and after that, left to stand in a fume hood overnight to form a film.

5. The film was removed from the teflon plate to complete the preparation of polycaprolactone/gelatin (PCL/Gelatin) film.

### Example 3: Preparation of Polycaprolactone/Poly(Lactic-Co-Glycolic Acid)/Gelatin (PCL/PLGA/Gelatin) Film

1. 3.2 g of polycaprolactone (Mw. 120K) and 0.8 g of poly(lactic-co-glycolic acid) were added to 20 ml of dichloromethane (DCM), and then mixed with it at 50 rpm for 3 hours to prepare a 20% polycaprolactone/poly(lactic-co-glycolic acid) solution.

2. 5 g of gelatin was added to 10 ml of deionized water and heated in a 50° C. oven for 16 hours to be dissolved to prepare a 50% gelatin solution.

3. The 50% gelatin solution was taken out from the oven and poured to the 20% polycaprolactone/poly(lactic-co-glycolic acid) solution (time for taking the 50% gelatin solution out and pouring it to the 20% polycaprolactone solution has to be in 1 minute) to perform mixing and stirring to form a mixture.

4. After stirring for about 1 minute±10 seconds, the mixture was poured on a teflon plate, and scraped with a 300 μm scraper to perform a film scraping procedure, and after that, left to stand in a fume hood overnight to form a film.

5. The film was removed from the teflon plate to complete the preparation of polycaprolactone/poly(lactic-co-glycolic acid)/gelatin (PCL/PLGA/Gelatin) film.

### B. Film Property Analysis

1. Uniformity Analysis for Films of Polycaprolactone Blended with Hydrophilic and/or Hydrophobic Polymer

Thermogravimetric Analysis (TGA)

Thermogravimetric analysis is often used to determine the properties of a substance by mass decrease or increase resulting from decomposition, oxidation or volatilization (such as volatilization of moisture content). Thermogravimetric analysis can be used to accurately predict material structure, or it can be directly used as a chemical analysis, and as a technique for observing blending uniformity.

A thermogravimetric analyzer used in the present experiment was Pyris 1 TGA. The procedure for operation and analysis is described in the following paragraphs.

The machine and computer were turned on, and it was confirmed that the machine was connected to the computer. The gas used was high-purity nitrogen, and it was confirmed that the nitrogen was sufficient and was led into the machine. “Pyris Manager” was clicked, and the thermogravimetric analysis software was started. The determining parameter conditions were set: Initial temperature was 25° C. Increasing the temperature to 700° C. at a rate of 20° C. per minute. Maintaining at 700° C. for 15 minutes. Information related to the file was filled out, such as storage location, file name, remark information, etc. According to the operation of the machine, a required platinum plate was hanged on a balance of the machine, a button for balancing was clicked to reset the weight of the platinum plate to zero. A temperature controlling barrier was lowered and the platinum plate was taken out and a sample to be tested was placed on the platinum plate, and the weight of the sample was controlled at 3-30 mg. A button for weighting was clicked to weigh the sample, and after the weight was determined, the measurement was begun. The result of the measurement was saved as an ASC file and analyzed.

First, the influence of a content of the hydrophilic polymer, alginate, on the uniformity of a film of polycaprolactone blended with hydrophilic and/or hydrophobic polymer was determined.

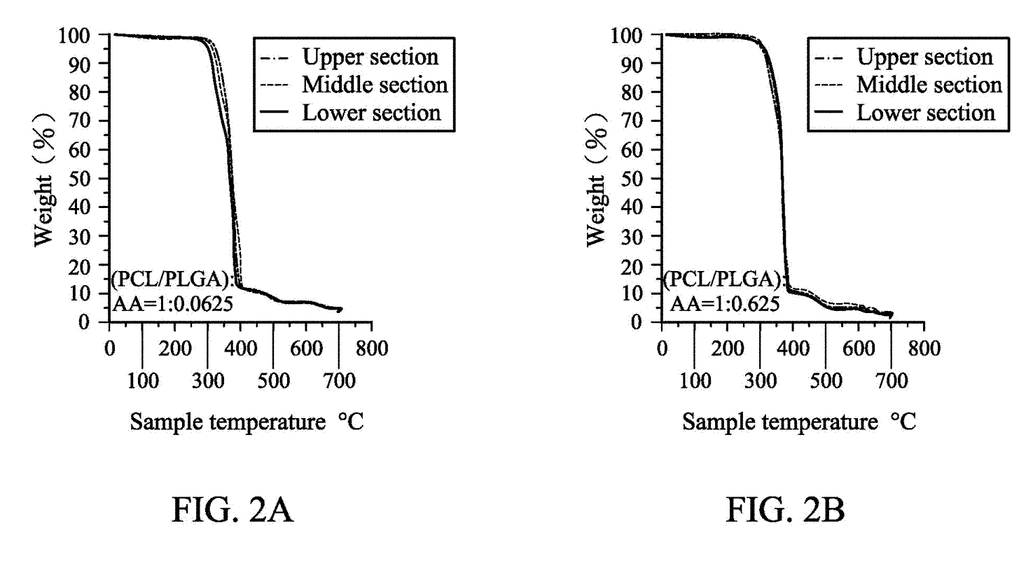

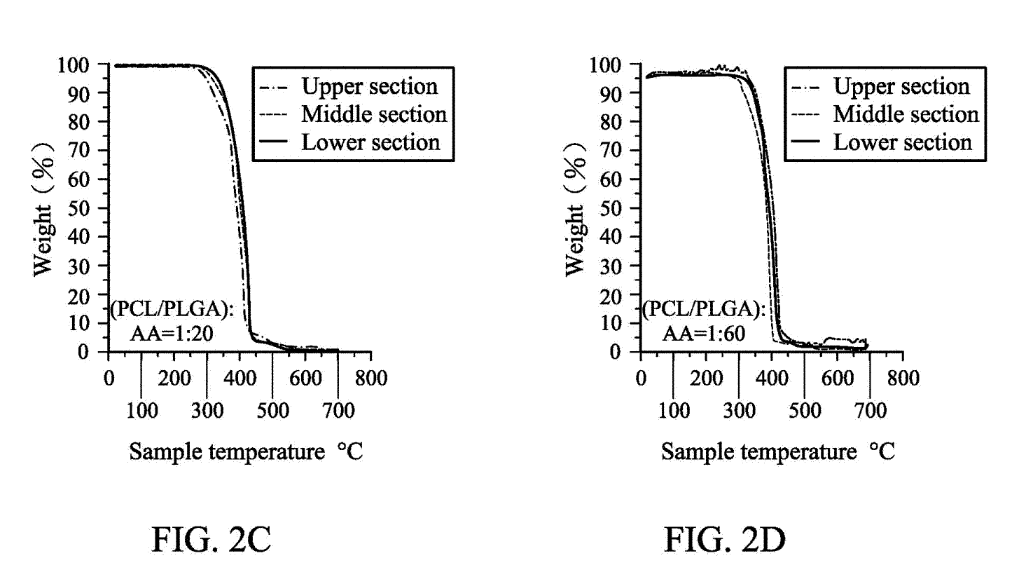

Films formed by blending different weights of alginate to a fixed weight of polycaprolactone/poly(lactic-co-glycolic acid) (the films prepared in Examples 1-1 to 1-4) were observed for composition uniformity at different locations (a film was cross cut into three sections: an upper section, a middle section, and a lower section) using a thermogravimetric analyzer. In the film prepared in Example 1-1, the weight ratio of (polycaprolactone/poly(lactic-co-glycolic acid)) to alginate is 1:0.0625. In the film prepared in Example 1-2, the weight ratio of (polycaprolactone/poly(lactic-co-glycolic acid)) to alginate is 1:0.625. In the film prepared in Example 1-3, the weight ratio of (polycaprolactone/poly(lactic-co-glycolic acid)) to alginate is 1:20. In the film prepared in Example 1-4, the weight ratio of (polycaprolactone/poly(lactic-co-glycolic acid)) to alginate is 1:60. The thermogravimetric analysis results for the films of Examples 1-1 to 1-4 are shown in FIGS. 2A to 2D, respectively.

The thermogravimetric analysis results showed that the films prepared in Example 1-2 was most uniform, i.e. when the weight ratio of the hydrophobic composition to the hydrophilic composition was 1:0.625, a film was most uniformly formed (FIG. 2B).

In addition, it was determined whether a hydrophilic polymer which was different from alginate in a film of polycaprolactone blended with hydrophilic and/or hydrophobic polymer was also capable of achieving the effect of uniformly forming a film.

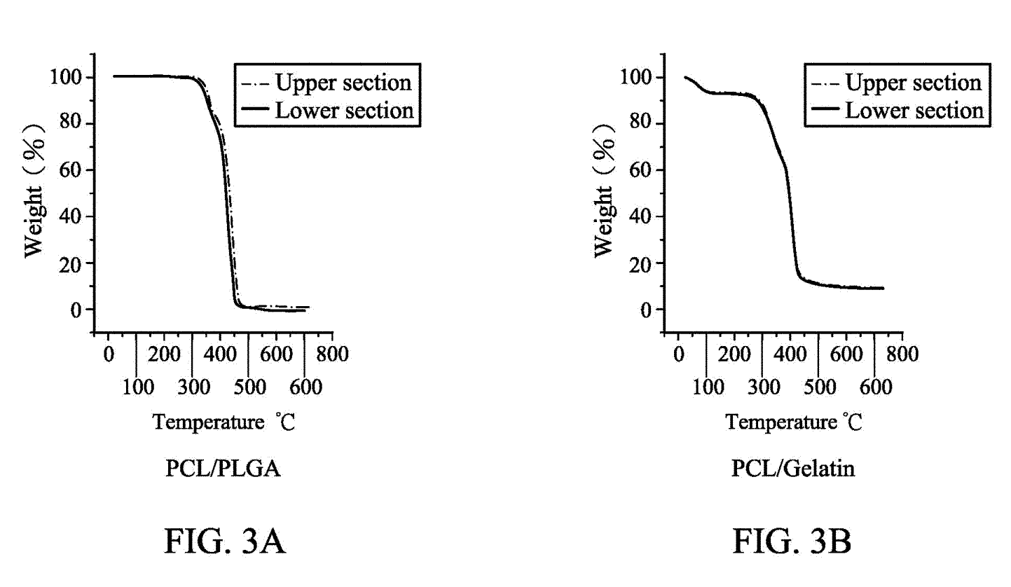

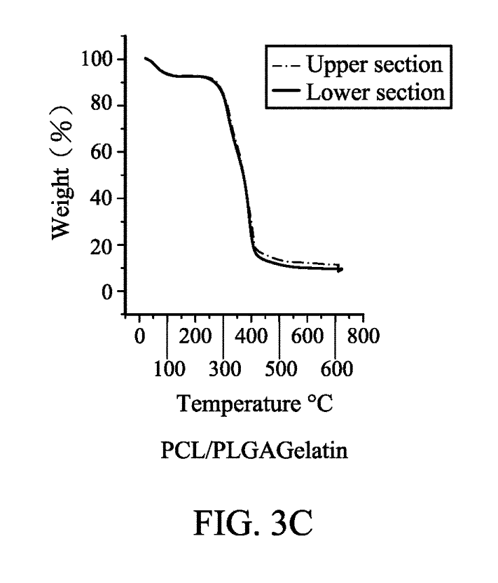

Films formed in Comparative Example 1 (polycaprolactone/poly(lactic-co-glycolic acid) film), Example 2 (polycaprolactone/gelatin film) and Example 3 (polycaprolactone/poly(lactic-co-glycolic acid)/gelatin) were observed for composition uniformity at different locations (a film was cross cut into two sections: an upper section and a lower section) using a thermogravimetric analyzer. The results are shown in FIGS. 3A to 3C.

FIGS. 3A to 3C show that the film prepared in Example 2 shows only one TGA curve while the film prepared in Example 3 shows two TGA curves which are completely overlapping. This indicated that by using the hydrophilic polymer, gelatin, as a dispersing agent, polycaprolactone can be uniformly blended with poly(lactic-co-glycolic acid) without phase separation occurring.

In contrast, the film prepared in Comparative Example 1 showed two obvious TGA curves. This indicated that the film formed without using a hydrophilic polymer as a dispersing agent was extremely nonuniform with phase separation occurring.

2. Fourier Transform Infrared Spectrometry (FT-IR) Analysis

The principle behind Fourier transform infrared spectrometry is that, for a molecule, when vibration-rotation occurs at various bonding structures in the molecule, the molecule absorbs appropriate infrared energy to obtain a spectrometry. Since infrared spectrometry can provide information about the properties of a molecular structure, and except for optical isomers, there is almost no identical spectrometry for different organic compounds, the structure and the properties of oscillating bonds or rotating bonds can be discerned by investigations of infrared spectrometry, and the presence and content of a compound can be identified or analyzed at the same time.

The operation procedure for Fourier transform infrared spectrometry analysis is described in the following paragraphs.

The film prepared in Example 3 (polycaprolactone/poly(lactic-co-glycolic acid)/gelatin) was cut to fit a size required by the stage of the equipment to be ready-for-use.

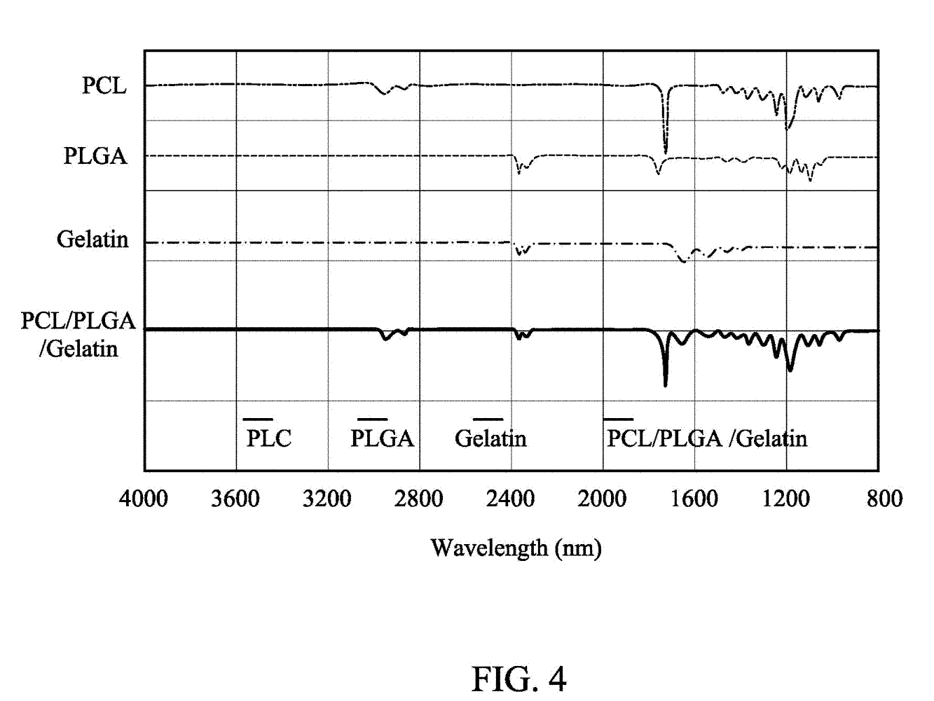

First, a sample stage cleaning was performed by wiping the sample stage with ethanol and leaving it to stand for 1 minute to let the ethanol volatilize. After the ethanol volatilized, the sample stage was pressed down to be fixed (without placing any matter thereon). The “Spectrometer setup” button of the software was clicked to enter a settings screen to determine that the laser intensity of the equipment was stable. After the determination, the “Background” button was clicked to detect the background level. After the detection, a file name was set and the whole working file was saved in a folder. The sample to be tested was placed on the sample stage, and if the sample was a film sample, the surface to be tested was face-down, and the stage was pressed down to be fixed to perform a detection. The “Scan” button in the “Scan” item in the toolbar was clicked to enter parameter settings, and “Scans” was set to 32, “Resolution” was set to 4, and “Truncation Range” was set to manual mode, and the range was set to 4000-800. Since the detecting chip of the sample stage had an absorbing effect on light with a wavelength less than 700, after removing the noise region, the range of 4000-800 was selected and the background level previously detected was deducted, and then the completed spectrometry file was saved and analyzed. The result is shown in FIG. 4.

According to FIG. 4, the characteristic peaks for polycaprolactone included 2945 cm⁻¹ (CH₂ characteristic peak), 1724 cm⁻¹ (C═O [[s]] characteristic peak), 2864 cm⁻¹ (CH₂ stretching) and 1242 cm⁻¹ (COC characteristic peak), characteristic peaks for poly(lactic-co-glycolic acid) include 1754 cm⁻¹ (C═O characteristic peak), 1184 cm⁻¹ (COC characteristic peak) and 1192 cm⁻¹ (CO characteristic peak). Characteristic peaks for gelatin included 1629 cm⁻¹ (—C(O)NH₂ characteristic peak, amide I) and 1523 cm⁻¹ (—C(O)NH₂ characteristic peak, amide II), and the peak for the amide group located at 1652 cm⁻¹. This peak indicted that gelatin randomly curled, and indicted bonds for α-helix structure.

Moreover, FIG. 4 showed that the characteristic peaks for polycaprolactone, poly(lactic-co-glycolic acid) and gelatin all appeared in the Fourier transform infrared spectrometry for the film prepared in Example 3 (polycaprolactone/poly(lactic-co-glycolic acid)/gelatin), and this indicated that polycaprolactone, poly(lactic-co-glycolic acid) and gelatin of the film prepared in Example 3 were blended without chemical crosslinking, and polycaprolactone, poly(lactic-co-glycolic acid) and gelatin were uniformly blended since the characteristic peaks for these three substances appeared in the same section of the film.

3. Physicochemical Properties for Films

(1) Standard Test of Burst Strength

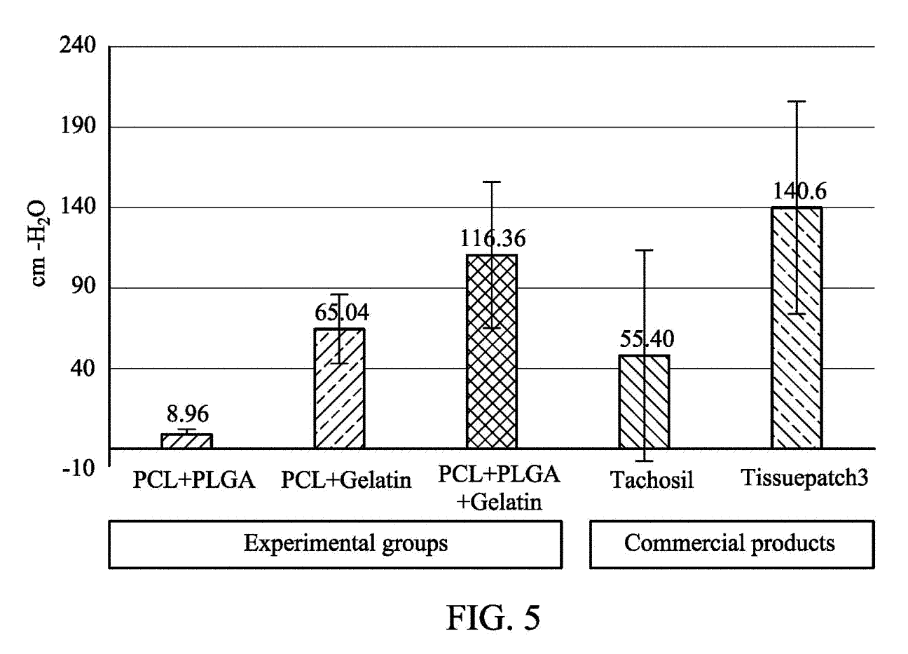

Films prepared in Comparative Example 1, Example 2 and Example 3, and the commercial sealing film (TachoSil) and sealing patch (TissePatch) (formed by polylactic acid (PLA), two layer structure, attachment effect is achieved by chemical covalent bonds) used clinically at present were attached to pig intestines, respectively, and a test of film burst strength was performed according to Standard Test Method for Burst Strength of Surgical Sealants) defined by ASTM F2392. The results are shown in FIG. 5.

FIG. 5 shows that the burst strength of the film prepared in Example 3 (polycaprolactone/poly(lactic-co-glycolic acid)/gelatin) is higher than that of the sealing film (TachoSil) in clinical use at present, and it matches that of the sealing patch (TissePatch) presently in clinical use.

(2) Test of Tensile Properties

Tensile tests were performed on the films prepared in Comparative Example 1, Example 2 and Example 3, and the commercial sealing film (TachoSil) used clinically at present and the sealing patch formed by polylactic acid (TissePatch) according to Standard Test Method for Tensile Properties of Thin Plastic Sheeting defined by ASTM D882-12.

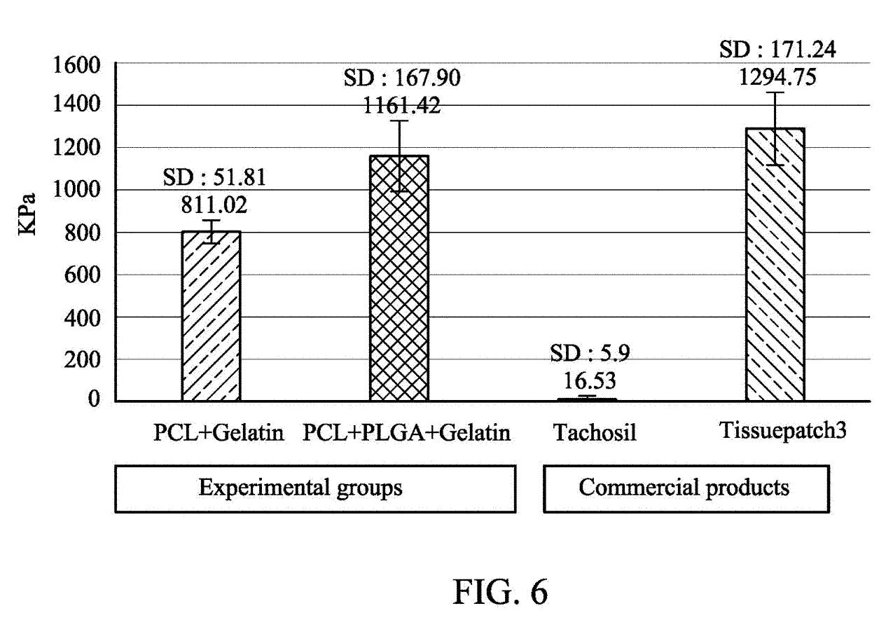

ASTM D882-12 standard is used to determine tensile properties, especially suitable for a plastic film with a thickness of less than 1 mm. Base on this standard, a test specimen was cut using a sharp cutter to a strip of 100*25.4 mm², and the initial distance between the upper and lower pneumatic chucks were adjusted to 100 mm, and the pulling speed was set to 50 mm/minute. The test results are shown in FIG. 6.

FIG. 6 shows that since the film prepared in Example 3 (polycaprolactone/poly(lactic-co-glycolic acid)/gelatin) combines the soft and perfect fitting properties of polycaprolactone with the mechanical strength of poly(lactic-co-glycolic acid), the tensile strength thereof is much higher than that of the sealing film (TachoSil) used clinically at present, and matches that of the sealing patch (TissePatch) used clinically at present.

3. Surface Structure and Roughness of Films

(1) Observation of Surface Structure

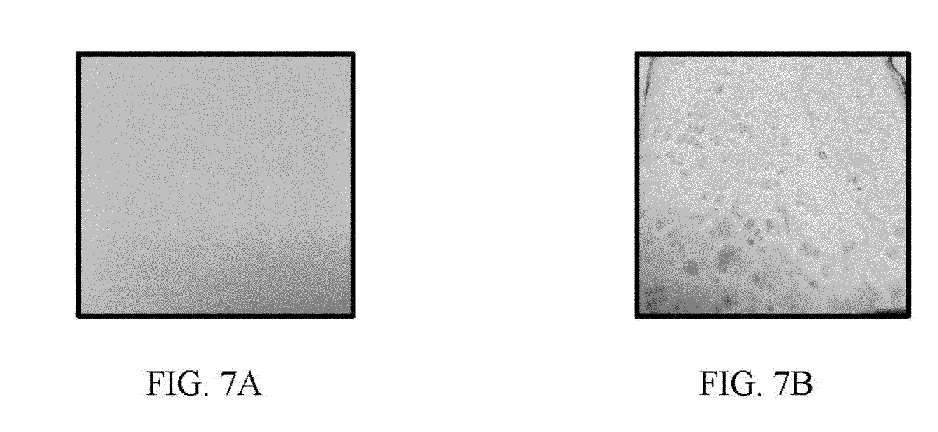

A film prepared using the method of the present disclosure (the film prepared in Example 3 (polycaprolactone/poly(lactic-co-glycolic acid)/gelatin)) and a film prepared without using the process of the present disclosure (the film prepared in Comparative Example 1 (polycaprolactone/poly(lactic-co-glycolic acid))) were observed with a microscope to perform a surface structure observation of the two films. The results are shown in FIGS. 7A and 7B.

The results show that the surface of the film prepared using the process of the present disclosure (the film prepared in Example 3 (polycaprolactone/poly(lactic-co-glycolic acid)/gelatin)) is uniform without phase separation or particles (FIG. 7A).

In contrast, there was serious phase separation on the surface of the film prepared without using the process of the present disclosure (the film prepared in Comparative Example 1 (polycaprolactone/poly(lactic-co-glycolic acid))), and the film could not even take shape (FIG. 7B).

(2) Roughness

Surface roughness of films prepared by the method of the present disclosure (the films prepared in Example 2 and Example 3) and a film prepared without using the process of the present disclosure (the film prepared in Comparative Example 1) were determined according to Standard Test Method for Surface Roughness defined by ASTM D7127-13.

Measurement was performed according to ASTM D7127-13 standard. The measuring instrument used was Surfcorder SE1700. The principle behind this measurement is that, by scanning the height difference of a test specimen with a probe through a light reflection and using scattering transmission, the reflective signals from the light sources which are on and under the probe were converted and calculated to draw a sectional drawing to determine the film thickness variation and surface roughness. After that, full roughness (Rz) was calculated.

Calculation for full roughness (Rz) is summarized in the following paragraph.

An estimated length was divided equally into 5 aliquots of a sample length, and the distance between the highest point and the lowest point in each aliquot of the sample length was calculated, and then the all the distances between the highest point and the lowest point in each aliquot of the sample length are summarized and averaged to obtain full roughness.

Formula for full roughness is shown in the following:

$$Rz = \frac{{R}_{y 1} + {R}_{y 2} + {R}_{y 3} + {R}_{y 4} + {R}_{y 5}}{5} ,$$

wherein R_{y1}, R_{y2}, R_{y3}, R_{y4} and R_{y5} respectively represent the distance between the highest point and the lowest point in the first aliquot to the fifth aliquot.

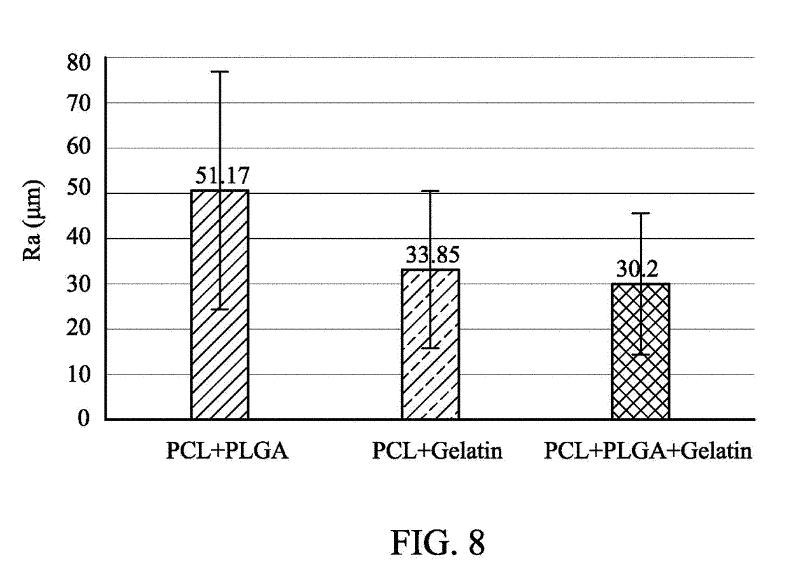

The results are shown in FIG. 8. Generally, in a condition without using a surfactant, a hydrophilic polymer and a hydrophobic polymer are not compatible, and that results in microphase separation and makes the film surface rough and uneven.

As shown in FIG. 8, the film prepared in Comparative Example 1 without using the process of the present disclosure (the film prepared in Comparative Example 1 (polycaprolactone/poly(lactic-co-glycolic acid))) still suffered from microphase separation due to the two polymers having different properties, and that resulted in surface microphase separation and a rough structure. In contrast, for the film prepared by the process in which gelatin was used as a dispersing agent of the present disclosure (the film prepared in Example 3 (polycaprolactone/poly(lactic-co-glycolic acid)/gelatin)), the gelatin in the film would not influence the roughness of the film surface, and no phase separation appeared in the hydrophilic polymer and the hydrophobic polymer in the film, giving the film surface a smooth and compact structure.

4. Animal Experiments

(1) Attachment Experiment for Rat Liver

First, Rompun and Zolite were mixed at a ratio of 1:1 to formulate an anesthetic. Anesthesia was administered to a rat with 0.4 ml/kg of anesthetic by intramuscular injection (IM) at the thigh muscles.

After the rat was deeply anaesthetized, hairs of the rat on the location to be subjected to a surgery were sheared. After that, the rat was placed on an aseptic operating table and covered with an aseptic hole-towel, and the location to be subjected to surgery was surface sterilized with povidone iodine.

The abdomen of the rat was cut to open and the position of the liver was found, and then a surface wound was made on the liver, and the size of the wound was about 1-1.5 cm.

Next, a film of the present disclosure was attached to the wound, forming a patch. After the attachment, the wound was allowed to stand for 30 seconds to confirm that the film was indeed attached to the wound. After that, abdominal muscles and epidermis of the rat were sutured. After suturing, the sutured positions were surface sterilized with povidone iodine to complete the surgery.

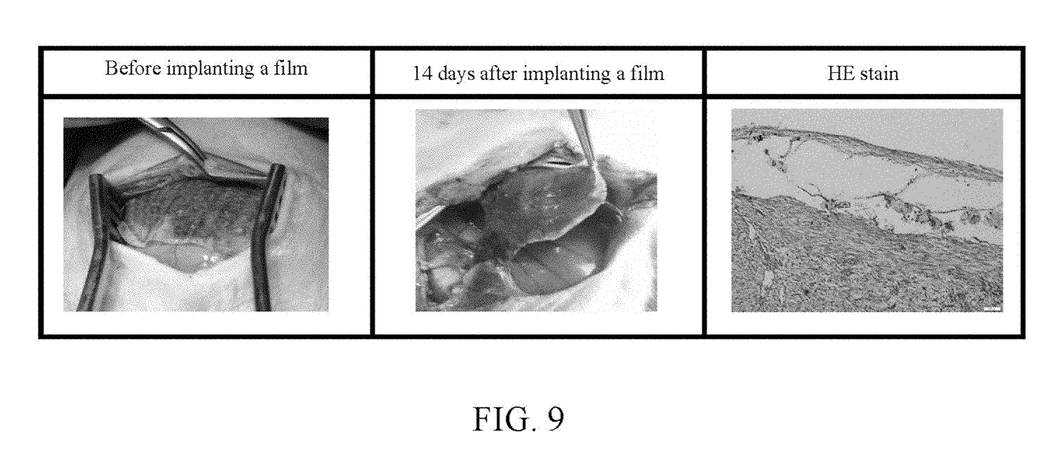

After the surgery, physiological observation was performed on the animal. 14 days after the film was implanted, the rat was sacrificed. After that, the appearance of the surgical site was observed and photographed, and the tissue to which the film was attached was sampled and a hematoxylin and eosin (H&E) stain was performed thereon. The results are shown in FIG. 9.

The results showed that 14 days after the surgery, the film was still capable of effectively attaching to the original surgical site.

(2) Animal Attachment Experiment for Rat Stomach

First, Rompun and Zolite were mixed at a ratio of 1:1 to formulate an anesthetic. Anesthesia was administered to a rat with 0.4 ml/kg of anesthetic by intramuscular injection (IM) at the thigh muscles.

After the rat was deeply anaesthetized, hairs of the rat on the location to be subjected to a surgery were sheared. After that, the rat was placed on an aseptic operating table and covered with an aseptic hole-towel, and the location to be subjected to surgery was surface sterilized with povidone iodine.

A laparotomy was performed on the rat, and the stomach was cut to make a perforation with a length of about 0.5 cm, and then in the middle of the perforation, i.e. in a position about 0.25 cm along the perforation length, a stitch was made using sutures to create a leaking wound. After that, a film of the present disclosure was attached directly to the leaking wound.

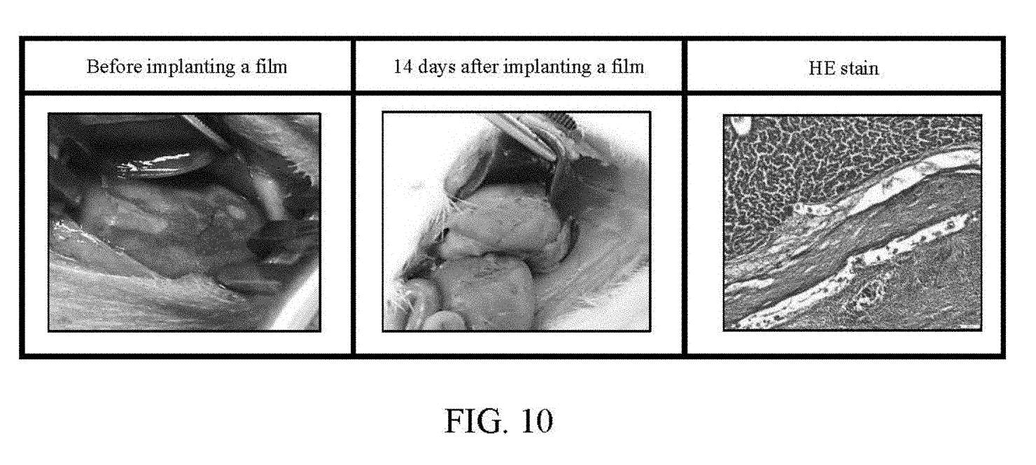

After the surgery, physiological observation was performed on the animal. 14 days after implanting the film, the rat was sacrificed. After that, the appearance of the surgical site was observed and photographed, and the tissue to which the film was attached was sampled and a hematoxylin and eosin (H&E) stain was performed thereon. The results are shown in FIG. 10.

According to FIG. 10, only through visual observation could it be confirmed that the film of the present disclosure was still at the original surgical site.

The results of the film patching experiment on the two animal organs showed that, after being attached to the liver and stomach, the films were not fixed with sutures.

However, 2 weeks after implanting the films of the present disclosure to a living body, it was confirmed through observation that the films of the present disclosure were still at the original surgical sites (see FIG. 9 and FIG. 10).

In addition, the results of the hematoxylin and eosin (H&E) stains also showed that the film would not induce a serious immune response and could promote tissue repair.

It will be apparent to those skilled in the art that various modifications and variations can be made to the disclosed embodiments. It is intended that the specification and examples be considered as exemplary only, with the true scope of the disclosure being indicated by the following claims and their equivalents.

1. A film, composed of a polymer mixture,
wherein the polymer mixture comprises:
a hydrophobic composition comprising polycaprolactone (PCL); and
at least one hydrophilic polymer, selected from a group consisting of:
alginate, gelatin, chitosan, hyaluronic acid, collagen, demineralized bone matrix (DBM), carboxymethyl cellulose (CMC), fibrin, polyoxyethylene, polyethylene glycol (PEG) and polyvinylpyrrolidone,
wherein a weight ratio of the hydrophobic composition to the at least one hydrophilic polymer is 1:0.01-100.

2. The film as claimed in claim 1, wherein a molecular weight of the polycaprolactone is 5000-150000.

3. The film as claimed in claim 1, wherein the at least one hydrophilic polymer is in the form of a solid particle.

4. The film as claimed in claim 3, wherein a particle size of the solid particle is 1-1000 μm.

5. The film as claimed in claim 1, wherein the at least one hydrophilic polymer is dissolved in a solvent to be in the form of a liquid polymer.

6. The film as claimed in claim 5, wherein the solvent may comprise water, ethanol, acetone, an acidic solution, an alkaline solution or a buffer solution.

7. The film as claimed in claim 5, wherein a limiting viscosity of the liquid polymer is 1-200 dl/g.

8. The film as claimed in claim 1, wherein the at least one hydrophilic polymer is alginate.

9. The film as claimed in claim 8, wherein the weight ratio of the polycaprolactone to the alginate is 1:0.05-80.

10. The film as claimed in claim 1, wherein the at least one hydrophilic polymer is gelatin.

11. The film as claimed in claim 10, wherein the weight ratio of the polycaprolactone to the gelatin is 1:0.05-80.

12. The film as claimed in claim 10, wherein the hydrophobic composition further comprises at least one hydrophobic polymer which is selected from a group consisting of polylactic acid (PLA), poly(lactic-co-glycolic acid) (PLGA), poly(glycolic acid) (PGA), polyhydroxybutyrate (PHB), polydioxanone (PDS), poly(propylene fumarate) (PPF), polyanhydrides, polyacetals, poly(ortho esters, polycarbonates, polyurethanes, polyphosphazenes and polyphosphoester.

13. The film as claimed in claim 12, wherein the at least one hydrophobic polymer is poly(lactic-co-glycolic acid).

14. The film as claimed in claim 13, wherein the molecular weight of the poly(lactic-co-glycolic acid) is 5000-150000.

15. The film as claimed in claim 13, wherein the at least one hydrophilic polymer is alginate.

16. The film as claimed in claim 15, wherein the weight ratio of the hydrophobic composition to the alginate is 1:0.05-80.

17. The film as claimed in claim 13, wherein the at least one hydrophilic polymer is gelatin.

18. The film as claimed in claim 17, wherein the weight ratio of the hydrophobic composition to the gelatin is about 1:0.05-80.

19. A method for manufacturing a film, comprising:
preparing a polymer mixture, wherein a method for preparing the polymer mixture comprises:
preparing a hydrophobic solution, wherein a solute of the hydrophobic solution comprises polycaprolactone; and
adding at least one hydrophilic polymer as a dispersing agent to the hydrophobic solution and mixing it with the hydrophobic solution, wherein the at least one hydrophilic polymer is selected from a group consisting of: alginate, gelatin, chitosan, hyaluronic acid, collagen, demineralized bone matrix (DBM), carboxymethyl cellulose (CMC), fibrin, polyoxyethylene, polyethylene glycol (PEG) and polyvinylpyrrolidone,
wherein the weight ratio of the solute of the hydrophobic solution to the at least one hydrophilic polymer is 1:0.01-100; and
drying the polymer mixture to form a film.

20. The method for manufacturing a film as claimed in claim 19, wherein the molecular weight of the polycaprolactone is 5000-150000.

21. The method for manufacturing a film as claimed in claim 19, wherein the hydrophobic solution is formed by dissolving the polycaprolactone in a solvent.

22. The method for manufacturing a film as claimed in claim 21, wherein the solvent comprises acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene or a mixture solution thereof.

23. The method for manufacturing a film as claimed in claim 19, wherein the at least one hydrophilic polymer is in the form of a solid particle.

24. The method for manufacturing a film as claimed in claim 23, wherein the particle size of the solid particle is 1-1000 μm.

25. The method for manufacturing a film as claimed in claim 19, wherein the at least one hydrophilic polymer is dissolved in a solvent to be in the form of a liquid polymer.

26. The method for manufacturing a film as claimed in claim 25, wherein the solvent may comprise water, ethanol, acetone, an acidic solution, an alkaline solution or a buffer solution.

27. The method for manufacturing a film as claimed in claim 25, wherein the limiting viscosity of the liquid polymer is about 1-200 dl/g.

28. The method for manufacturing a film as claimed in claim 19, wherein the at least one hydrophilic polymer is alginate.

29. The method for manufacturing a film as claimed in claim 28, wherein the weight ratio of the polycaprolactone to the alginate is 1:0.05-80.

30. The method for manufacturing a film as claimed in claim 19, wherein the at least one hydrophilic polymer is gelatin.

31. The method for manufacturing a film as claimed in claim 30, wherein the weight ratio of the polycaprolactone to the gelatin is 1:0.05-80.

32. The method for manufacturing a film as claimed in claim 19, wherein the solute of the hydrophobic solution further comprises a hydrophobic polymer and the hydrophobic polymer is selected from a group consisting of polylactic acid (PLA), poly(lactic-co-glycolic acid) (PLGA), poly(glycolic acid) (PGA), polyhydroxybutyrate (PHB), polydioxanone (PDS), polypropylene fumarate) (PPF), polyanhydrides, polyacetals, poly(ortho esters, polycarbonates, polyurethanes, polyphosphazenes and polyphosphoester.

33. The method for manufacturing a film as claimed in claim 32, wherein the hydrophobic solution is formed by a method which comprises:
dissolving the polycaprolactone in a first solvent to form a first solution, and dissolving the at least one hydrophobic polymer in a second solvent to form a second solution, wherein the first solvent and the second solvent are the same or different; and
mixing the first solution with the second solution to form the hydrophobic solution.

34. The method for manufacturing a film as claimed in claim 33, wherein the first solvent and the second solvent independently comprises acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene or a mixture solution thereof.

35. The method for manufacturing a film as claimed in claim 32, wherein the hydrophobic solution is formed by a method which comprises:
dissolving the polycaprolactone and the at least one hydrophobic polymer in a solvent to form the hydrophobic solution.

36. The method for manufacturing a film as claimed in claim 35, wherein the solvent comprises acetone, acetic acid, chloroform, methanol, dichloromethane, dimethylformamide, dioxane, ethyl acetate, formic acid, hexafluoroisopropanol, 1-methyl-2-pyrrolidone, tetrahydrofuran, toluene or a mixture solution thereof.

37. The method for manufacturing a film as claimed in claim 32, wherein the at least one hydrophobic polymer is poly(lactic-co-glycolic acid).

38. The method for manufacturing a film as claimed in claim 37, wherein the molecular weight of the poly(lactic-co-glycolic acid) is 5000-150000.

39. The method for manufacturing a film as claimed in claim 37, wherein the at least one hydrophilic polymer is alginate.

40. The method for manufacturing a film as claimed in claim 39, wherein the weight ratio of the solute of the hydrophobic solution to the alginate is 1:0.05-80.

41. The method for manufacturing a film as claimed in claim 37, wherein the at least one hydrophilic polymer is gelatin.

42. The method for manufacturing a film as claimed in claim 41, wherein the weight ratio of the solute of the hydrophobic solution to the gelatin is 1:0.05-80.

43. A method for sealing a surgical wound or a diffuse wound, comprising:
applying the film as claimed in claim 1 as a laminating film to a surgical wound or a diffuse wound to attach to the surgical wound or the diffuse wound.

44. The method for sealing a surgical wound or a diffuse wound as claimed in claim 43, wherein the hydrophobic composition further comprises at least one hydrophobic polymer which is selected from a group consisting of polylactic acid (PLA), poly(lactic-co-glycolic acid) (PLGA), poly(glycolic acid) (PGA), polyhydroxybutyrate (PHB), polydioxanone (PDS), poly(propylene fumarate) (PPF), polyanhydrides, polyacetals, poly(ortho esters, polycarbonates, polyurethanes, polyphosphazenes and polyphosphoester.
